SEQUENCE LISTING (10 sequences)

SEQ ID NO: 1 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 344 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 345 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 5 aa, synthetic construct
  REGION 1..5: note = Synthetic
GGGGS

SEQ ID NO: 10 - protein, 11 aa, synthetic construct
  REGION 1..11: note = Synthetic
GTELSVRAKPS

(19) United States
(12) Patent Application Publication
(10) Pub. No.: US 2025/0345395 A1
(43) Pub. Date: Nov. 13, 2025

(54) Dosing Regimens of SIRP Alpha Fusion Proteins for Treatment of Cancer

(71) Applicant: PFIZER INC., NEW YORK, NY (US)

(72) Inventors: Margaret Victoria Elizabeth ALLGOOD, Whiteville, NC (US); Ingmar BRUNS, Cambridge, MA (US); Victor Ruberio LINCHA, San Diego, CA (US); Dmitri PAVLOV, San Diego, CA (US); Anita SCHEUBER, Boston, MA (US); Diane Dan WANG, San Diego, CA (US); Yibo WANG, Ellicott City, MD (US)

(73) Assignee: PFIZER INC., NEW YORK, NY (US)

(21) Appl. No.: 18/868,668

(22) PCT Filed: May 22, 2023
(86) PCT No.: PCT/IB2023/055236

§ 371 Date: Nov. 22, 2024

Related U.S. Application Data
(60) Provisional application No. 63/345,693, filed on May 25, 2022.
(60) Provisional application No. 63/492,121, filed on Mar. 24, 2023.

(51) Int. Cl. A61K 38/17; A61K 39/00; A61K 39/395; A61P 35/00; C07K 14/705; C07K 16/28

(52) U.S. Cl. CPC A61K 38/177; A61K 39/39558; A61P 35/00; C07K 14/705; C07K 16/2887; A61K 2039/545; C07K 2319/30

(57) ABSTRACT

Dosing regimens and methods for administering SIRPaFc fusion proteins are provided. The dosing regimens and methods include both SIRPaFc monotherapies and combination therapies.

### BACKGROUND

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα to treat cancer by CD47 blockade is described in WO 2010/130053, incorporated herein by reference.

International Patent Application Publication No. WO 2014/094122, incorporated by reference in its entirety, describes a protein drug that inhibits the interaction between CD47 and SIRPα. This CD47 blockade drug is a form of human SIRPα that incorporates a particular region of its extracellular domain linked with a particularly useful form of an IgG-based Fc region. In this form, the SIRPαFc drug shows dramatic effects on the viability of cancer cells that present with a CD47+ phenotype. The effect is seen particularly on acute myelogenous leukemia (AML) cells, and on many other types of cancer.

The CD47 blockade approach in anti-cancer drug development shows great promise. However, improved dosing regimens and treatment methods for SIRPαFc agents are needed.

### SUMMARY

Provided herein are improved dosage regimens and treatment methods for SIRPaFc-based therapies. Dosing regimens and methods provided herein include both SIRPaFc monotherapies and combination therapies.

In some embodiments, provided herein is a method of treating a cancer in a patient, the method comprising administering a SIRPαFc fusion protein to the patient according to a dosing regimen of 8 mg/kg, 10 mg/kg, 12 mg/kg, 16 mg/kg, 18 mg/kg, 24 mg/kg, 28 mg/kg, 300 mg, 600 mg, 1200 mg, 1500 mg, 1800 mg, 2100 mg, or 2400 mg, Q1W, Q2W, or Q3W.

In some embodiments provided herein is a method of treating a cancer in a patient, comprising administering a SIRPaFc fusion protein to the patient according to a dosing regimen of 8 mg/kg Q1W for 4 weeks followed by 18 mg/kg Q3W.

In some embodiments, provided herein is a method of treating a cancer in a patient, comprising administering a SIRPaFc fusion protein to the patient according to a dosing regimen of 16 mg/kg Q1W for 4 weeks followed by 28 mg/kg Q3W.

In some embodiments, provided herein is a method of treating a cancer in a patient, comprising administering a SIRPaFc fusion protein to the patient according to a dosing regimen of 8 mg/kg Q1W, 18 mg/kg Q3W, 16 mg/kg QW, or 28 mg/kg Q3W.

In some embodiments, provided herein is a method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 8 mg/kg Q1W for 4 weeks followed by 18 mg/kg Q3W.

In some embodiments, provided herein is a method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 16 mg/kg Q1W for 4 weeks followed by 28 mg/kg Q3W.

In some embodiments, provided herein is a method of treating a cancer in a patient, comprising administering a SIRPaFc fusion protein to the patient according to a dosing regimen of 8 mg/kg Q1W, 16 mg/kg Q1W, or 10 mg/kg Q2W.

In some embodiments, provided herein is a method of treating a cancer in a patient, the method comprising administering a combination therapy of a SIRPaFc fusion protein, carfilzomib, and dexamethasone to the patient for N cycles, wherein each cycle is 28 days and the SIRPaFc fusion protein is administered at 8 mg/kg or 16 mg/kg on days 1, 8, 15, and 22 of the 28 day cycle, carfilzomib is administered at 20 mg/m2 or 70 mg/mg2 on days 1, 8, and 15 of the 28 day cycle, dexamethasone is administered at 40 mg on days 1, 8, 15, and 22 of the 28 day cycle.

In some embodiments, provided herein is a method of treating a cancer in a patient, the method comprising administering a combination therapy of a SIRPaFc fusion protein, carfilzomib, and dexamethasone to the patient for N cycles, wherein each cycle is 28 days and the SIRPaFc fusion protein is administered at 10 mg/kg on days 1 and 15 of the 28 day cycle, carfilzomib is administered at 20 mg/m2 or 70 mg/mg2 on days 1, 8, and 15 of the 28 day cycle, dexamethasone is administered at 40 mg on days 1, 8, 15, and 22 of the 28 day cycle.

In some embodiments, provided herein is a method of treating a cancer in a patient, comprising administering a SIRPaFc fusion protein to the patient according to a dosing regimen of 0.2 mg/kg, 0.7 mg/kg, or 2.0 mg/kg Q2W.

In some embodiments, provided herein is a method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 2, 3, 4, 5, 6, 7, or 8 cycles, and wherein the second regimen follows the first regimen and comprises administering a SIRPaFc fusion protein to the patient according to a dosing regimen of Q2W.

In some embodiments, provided herein is a method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 2, 3, 4, 5, 6, 7, or 8 cycles, and wherein the second regimen follows the first regimen and comprises administering a SIRPaFc fusion protein to the patient according to a dosing regimen of Q2W.

In some embodiments, provided herein is a method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 6 cycles, and wherein the second regimen follows the first regimen and comprises administering a SIRPaFc fusion protein to the patient according to a dosing regimen of Q2W, wherein the doxorubicin is administered in the first regimen at a fixed dose of 75 mg/m2 and wherein the SIRPaFc fusion protein is administered in each of the first regimen and second regimen at a dose of 0.2 mg/kg, 0.7 mg/kg, or 2.0 mg/kg.

### BRIEF DESCRIPTION OF THE DRAWINGS

FIG. 1 depicts an exemplary SIRPaFc fusion protein dosing regimen containing a SIRPaFc fusion protein (TTI-622) and an anti-CD20 agent.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. A method of treating a cancer in a patient, comprising administering a SIRPaFc fusion protein to the patient according to a dosing regimen of 8 mg/kg, 10 mg/kg, 12 mg/kg, 16 mg/kg, 18 mg/kg, 24 mg/kg, 28 mg/kg, 150 mg, 300 mg, 600 mg, 1200 mg, or 2400 mg Q1W, Q2W, Q3W, or Q4W.

E2. A method of treating a cancer in a patient, comprising administering a SIRPaFc fusion protein to the patient according to a dosing regimen of 8 mg/kg Q1W for 4 weeks followed by 18 mg/kg Q3W.

E3. A method of treating a cancer in a patient, comprising administering a SIRPaFc fusion protein to the patient according to a dosing regimen of 16 mg/kg Q1W for 4 weeks followed by 28 mg/kg Q3W.

E4. A method of treating a cancer in a patient, comprising administering a SIRPaFc fusion protein to the patient according to a dosing regimen of 8 mg/kg Q1W, 18 mg/kg Q3W, 16 mg/kg QW, or 28 mg/kg Q3W.

E5. The method of any one of E1-E4 further comprising administering an anti-CD20 agent to the patient.

E6. A method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 8 mg/kg Q1W for 4 weeks followed by 18 mg/kg Q3W.

E7. A method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 16 mg/kg Q1W for 4 weeks followed by 28 mg/kg Q3W.

E8. The method of any one of E5-E7 wherein the anti-CD20 agent is rituximab

E9. A method of treating a cancer in a patient, comprising administering a SIRPaFc fusion protein to the patient according to a dosing regimen of 8 mg/kg Q1W, 16 mg/kg Q1W, or 10 mg/kg Q2W.

E10. The method of E9 further comprising administering carfilzomib and dexamethasone to the patient, optionally wherein the carfilzomib is administered by IV and the dexamethasone is administered by IV or orally.

E11. A method of treating a cancer in a patient, the method comprising administering a combination therapy of a SIRPaFc fusion protein, carfilzomib, and dexamethasone to the patient for N cycles, wherein each cycle is 28 days and the SIRPaFc fusion protein is administered at 8 mg/kg or 16 mg/kg on days 1, 8, 15, and 22 of the 28 day cycle, carfilzomib is administered at 20 mg/m2 or 70 mg/mg2 on days 1, 8, and 15 of the 28 day cycle, dexamethasone is administered at 40 mg on days 1, 8, 15, and 22 of the 28 day cycle.

E12. A method of treating a cancer in a patient, the method comprising administering a combination therapy of a SIRPaFc fusion protein, carfilzomib, and dexamethasone to the patient for N cycles, wherein each cycle is 28 days and the SIRPaFc fusion protein is administered at 10 mg/kg on days 1 and 15 of the 28 day cycle, carfilzomib is administered at 20 mg/m2 or 70 mg/mg2 on days 1, 8, and 15 of the 28 day cycle, dexamethasone is administered at 40 mg on days 1, 8, 15, and 22 of the 28 day cycle.

E13. The method of any one of E11 or E12, wherein N is 1, 2, 3, 4, 5, 6, 7, or 8 cycles.

E14. A method of treating a cancer in a patient, comprising administering a SIRPaFc fusion protein to the patient according to a dosing regimen of 0.2 mg/kg, 0.7 mg/kg, or 2.0 mg/kg Q2W.

E15. A method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 2, 3, 4, 5, 6, 7, or 8 cycles, and wherein the second regimen follows the first regimen and comprises administering a SIRPaFc fusion protein to the patient according to a dosing regimen of Q2W.

E16. The method of E15, wherein the doxorubicin is administered in the first regimen at a fixed dose of 75 mg/m2 and wherein the SIRPaFc fusion protein is administered in each of the first regimen and second regimen at a dose of 0.2 mg/kg, 0.7 mg/kg, or 2.0 mg/kg.

E17. A method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 6 cycles, and wherein the second regimen follows the first regimen and comprises administering a SIRPaFc fusion protein to the patient according to a dosing regimen of Q2W, wherein the doxorubicin is administered in the first regimen at a fixed dose of 75 mg/m2 and wherein the SIRPaFc fusion protein is administered in each of the first regimen and second regimen at a dose of 0.2 mg/kg, 0.7 mg/kg, or 2.0 mg/kg.

E18. A method of treating a cancer in a patient, comprising administering a SIRPaFc fusion protein to the patient according to a dosing regimen of 8 mg/kg QW.

E19. A method of treating a cancer in a patient, comprising administering a SIRPaFc fusion protein to the patient according to a dosing regimen of 16 mg/kg QW.

E20. A method of treating a cancer in a patient, comprising administering a SIRPaFc fusion protein to the patient according to a dosing regimen of 24 mg/kg QW.

E21. A method of treating a cancer in a patient, comprising administering a SIRPaFc fusion protein to the patient according to a dosing regimen of 10 mg/kg Q2W.

E22. A method of treating a cancer in a patient, comprising administering a SIRPaFc fusion protein to the patient according to a dosing regimen of 18 mg/kg Q3W.

E23. A method of treating a cancer in a patient, comprising administering a SIRPaFc fusion protein to the patient according to a dosing regimen of 28 mg/kg Q3W.

E24. A method of treating a cancer in a patient, comprising administering a SIRPaFc fusion protein to the patient according to a dosing regimen of 12 mg/kg QW.

E25. A method of treating a cancer in a patient, comprising administering a SIRPaFc fusion protein to the patient according to a dosing regimen of 12 mg/kg Q2W.

E26. A method of treating a cancer in a patient, comprising administering a SIRPaFc fusion protein to the patient according to a dosing regimen of 18 mg/kg QW.

E27. A method of treating a cancer in a patient, comprising administering a SIRPaFc fusion protein to the patient according to a dosing regimen of 18 mg/kg Q2W.

E28. A method of treating a cancer in a patient, comprising administering a SIRPaFc fusion protein to the patient according to a dosing regimen of 24 mg/kg QW.

E29. A method of treating a cancer in a patient, comprising administering a SIRPaFc fusion protein to the patient according to a dosing regimen of 24 mg/kg Q2W.

E30. A method of treating a cancer in a patient, comprising administering a SIRPaFc fusion protein to the patient according to a dosing regimen of 24 mg/kg Q3W.

E31. A method of treating a cancer in a patient, comprising administering a SIRPaFc fusion protein to the patient according to a dosing regimen of 32 mg/kg QW.

E32. A method of treating a cancer in a patient, comprising administering a SIRPaFc fusion protein to the patient according to a dosing regimen of 32 mg/kg Q2W.

E33. A method of treating a cancer in a patient, comprising administering a SIRPaFc fusion protein to the patient according to a dosing regimen of 32 mg/kg Q3W.

E34. A method of treating a cancer in a patient, comprising administering a SIRPaFc fusion protein to the patient according to a dosing regimen of 150 mg fixed dose, 300 mg fixed dose, 600 mg fixed dose, 900 mg fixed dose, 1200 mg fixed dose, 1500 mg fixed dose, 1800 mg fixed dose, 2100 mg fixed dose, or 2400 mg fixed dose Q1W.

E35. A method of treating a cancer in a patient, comprising administering a SIRPaFc fusion protein to the patient according to a dosing regimen of 150 mg fixed dose, 300 mg fixed dose, 600 mg fixed dose, 900 mg fixed dose, 1200 mg fixed dose, 1500 mg fixed dose, 1800 mg fixed dose, 2100 mg fixed dose, or 2400 mg fixed dose Q2W.

E36. A method of treating a cancer in a patient, comprising administering a SIRPaFc fusion protein to the patient according to a dosing regimen of 150 mg fixed dose, 300 mg fixed dose, 600 mg fixed dose, 900 mg fixed dose, 1200 mg fixed dose, 1500 mg fixed dose, 1800 mg fixed dose, 2100 mg fixed dose, or 2400 mg fixed dose Q3W.

E37. The method of any one of E1-E36, wherein the SIRPaFc fusion protein comprises a SIRPa polypeptide comprising the amino acid sequence of SEQ ID NO: 1.

E38. The method of any one of E1-E37, wherein the SIRPaFc fusion protein comprises a SIRPa polypeptide comprising the amino acid sequence of SEQ ID NO: 2.

E39. The method of any one of E1-E38, wherein the SIRPaFc fusion protein comprises the amino acid sequence of SEQ ID NO: 7 or SEQ ID NO: 8.

E40. The method of any one of E1-E13 or E18-E38, wherein the SIRPaFc fusion protein comprises the amino acid sequence of SEQ ID NO: 8.

E41. The method of any one of E14-E17, wherein the SIRPaFc fusion protein comprises the amino acid sequence of SEQ ID NO: 7.

E42. The method of any one of E1-E37, wherein the SIRPaFc fusion protein comprises a SIRPα polypeptide comprising the amino acid sequence of SEQ ID NO: 1 or a variant thereof having one, two, three, four, or five amino acid substitutions as compared the sequence of SEQ ID NO: 1.

E43. The method of any one of E1-E42, wherein the cancer is a blood cancer or a solid tumor cancer.

E44. The method of any one of E1-E43, wherein the cancer is selected from the group consisting of acute lymphocytic leukemia (ALL); acute myeloid leukemia (AML) and p53 mutated AML; chronic lymphocytic leukemia (CLL); chronic myelogenous leukemia (CML); myeloproliferative disorder/neoplasm (MPDS); myelodysplastic syndrome, lymphoma, T cell lymphoma, Hodgkin's lymphoma, indolent non-Hodgkin's lymphoma, aggressive non-Hodgkin's lymphoma, Burkitt's lymphoma, small cell follicular lymphoma, large cell follicular lymphoma. myeloma, multiple myeloma (MM), giant cell myeloma, heavy-chain myeloma, light chain or Bence-Jones myeloma, sarcoma, soft tissue sarcoma, leiomyosarcoma (LMS), undifferentiated pleomorphic sarcoma, myxofibrosarcoma, dedifferentiated liposarcoma, angiosarcoma, or epithelioid sarcoma.

E45. The method of any one of E1-E44, wherein the SIRPaFc fusion protein is administered for 12 doses or fewer.

E46. The method of any one of E1-E45, wherein the SIRPaFc fusion protein is administered until disease progression.

E47. The method of any one of E1-E46, wherein the patient has CD47-positive cancer cells.

E48. The method of any one of E1-E47, wherein the SIRPaFc fusion protein is administered subcutaneously (SC) or by intravenously (IV).

E49. A SIRPaFc fusion protein for use to treat a patient according to the method of any one of E1-E48.

E50. Use of a SIRPaFc fusion protein in the manufacture of a medicament for use to treat a patient according to the method of any one of E1-E48.

E51. A kit comprising a SIRPaFc fusion protein and instructions for use according to the method of any one of E1-E48.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of SIRPaFc fusion protein) means that the parameter may vary by as much as 10% below or above the stated numerical value for that parameter. For example, a dose of about 5 mg means 5%±10%, i.e. it may vary between 4.5 mg and 5.5 mg.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is cancer.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and (3) ameliorating the disease; for example, ameliorating a disease, condition or disorder in an individual that is experiencing or displaying the pathology or symptomatology of the disease, condition or disorder (i.e., reversing the pathology or symptomatology).

### SIRPaFc Fusion Protein Dosing Regimens

The present invention provides improved SIRPalpha-Fc (“SIRPaFc”) fusion protein dosing regimens and treatment methods. In the dosing regimens and methods provided herein, the SIRPaFc fusion protein may be administered as a monotherapy, or it may be administered in combination with one, two, or more additional therapeutic agents.

Dosing regimens and methods provided herein use a CD47-binding and blocking form of SIRPα, as a CD47 blockade drug or blocking agent. An agent or drug that has CD47 blockade activity is an agent that interferes with and dampens signal transmission that results when CD47 interacts with macrophage-presented SIRPα. CD47-binding forms of human SIRPα are the preferred CD47 blockade drugs for use in the regimens and methods provided herein. These drugs are based on the extracellular region of human SIRPα. They comprise at least a region of the extracellular region sufficient to confer effective CD47 binding affinity and specificity. So-called “soluble” forms of SIRPα, lacking the membrane anchoring component, are described in the literature and include those referenced in WO 2010/070047 (Novartis), WO2013/109752 (Stanford), and WO2014/094122 (Trillium), each incorporated by reference in its entirety.

In a preferred embodiment, the soluble form of SIRPα is an Fc fusion. More particularly, the drug suitably comprises the human SIRPα protein, in a form fused directly, or indirectly, with an antibody constant region, or Fc (fragment crystallisable). Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. The present invention is directed most particularly to the drug combinations that include the human SIRP variant 2 form, or V2.

In the dosing regimens and methods provided herein, useful SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1.

[SEQ ID NO: 1]
EELQVIQPDKSVSVAAGESAILHCTVTSLIPVGPIQWFRGAGPARELIY
NQKEGHFPRVTTVSESTKRENMDFSISISNITPADAGTYYCVKFRKGSP
DTEFKSGA

In some embodiments, SIRPαFc fusion proteins incorporate the IgV domain as defined by SEQ ID NO: 1, and additional, flanking residues contiguous within the SIRPα sequence. This form of the IgV domain, represented by residues 31-148 of the V2 form of human SIRPα, is a 118-mer having SEQ ID NO: 2 shown below:

[SEQ ID NO: 2]
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPS

The present SIRPα fusion proteins can also incorporate an Fc region having effector function. Fc refers to “fragment crystallisable” and represents the constant region of an antibody comprised principally of the heavy chain constant region and components within the hinge region. Suitable Fc components include those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In some embodiments, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 3:

[SEQ ID NO: 3]
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

Thus, in some embodiments, the Fc region has either a wild type or consensus sequence of an IgG1 constant region. In alternative embodiments, the Fc region incorporated in the fusion protein is derived from any IgG1 antibody having atypical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In some embodiments, the Fc region is based on the amino acid sequence of a human IgG4 set out as P01861 in UniProtKB/Swiss-Prot, residues 99-327, and has the amino acid sequence shown below and referenced herein as SEQ ID NO: 4:

[SEQ ID NO: 4]
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In some embodiments, the Fc region incorporates one or more alterations, usually not more than about 10, e.g., up to 1, 2, 3, 4, 5 or 6 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. 62/777,375, and many others. Particularly useful are those alterations that enhance Fc properties while remaining silent with respect to conformation, e.g., retaining Fc receptor binding. In another embodiment, the Fc region is modified to increase its biological half-life. Various approaches are possible. For example, one or more of the following mutations can be introduced; T252L, T254S, T256F, as described in U.S. Pat. No. 6,277,375.

In a specific embodiment, and in the case where the Fc component is an IgG4 Fc, the Fc incorporates at least the S²²⁸P mutation, and has the amino acid sequence set out below and referenced herein as SEQ ID NO: 5:

[SEQ ID NO: 5]
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The CD47 blockade drug used in the regimens and method provided herein is thus preferably a SIRP fusion protein useful to inhibit the binding of human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG having effector function.

In one embodiment, the fusion protein comprises a SIRPα component consisting at least of residues 32-137 of the V2 form of wild type human SIRPα, i.e., SEQ ID NO: 2. In a preferred embodiment, the SIRPα component consists of residues 31-148 of the V2 form of human SIRPα, i.e., SEQ ID NO: 2. In another embodiment, the Fc component is the Fc component of the human IgG1 designated P01857, and in a specific embodiment has the amino acid sequence that incorporates the lower hinge-CH2-CH3 region thereof i.e., SEQ ID NO: 3.

In some embodiments, the SIRPαFc fusion protein is provided and used in a secreted dimeric fusion form, wherein the fusion protein incorporates a SIRPα component having SEQ ID NO: 1 and preferably SEQ ID NO: 2 and, fused therewith, an Fc region having effector function and having SEQ ID NO: 3. When the SIRPα component is SEQ ID NO: 1, this fusion protein comprises SEQ ID NO: 6, shown below:

[SEQ ID NO: 6]
EELQVIQPDKSVSVAAGESAILHCTVTSLIPVGPIQWFRGAGPARELIY
NQKEGHFPRVTTVSESTKRENMDFSISISNITPADAGTYYCVKFRKGSP
DTEFKSGAGTELSVRAKPSDKTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K

When the SIRPα component is SEQ ID NO: 2, this fusion protein comprises SEQ ID NO: 7, shown below:

[SEQ ID NO: 7]
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

The SIRPaFc fusion protein of SEQ ID NO: 7 is also known as TTI-621.

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 2, the resulting IgG4-based SIRPα-Fc protein has SEQ ID NO: 8, shown below:

[SEQ ID NO: 8]
EEELQVIQPDKSVSVAAGESAILHCTVTSLIPVGPIQWFRGAGPARELI
YNQKEGHFPRVITVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

The SIRPaFc fusion protein of SEQ ID NO: 8 is also known as TTI-622.

In one embodiment of a dosing regimen or method provided herein, a SIRPaFc fusion protein comprises, as the SIRPα component of the fusion protein, a sequence that comprises SEQ ID NO: 2. In one embodiment, the SIRPaFc fusion protein comprises the polypeptide of SEQ ID NO: 7 or SEQ ID NO: 8.

The SIRPα sequence incorporated within the SIRPaFc fusion protein can be varied, as described in the literature. This can eliminate glycosylation sites in the protein, such as at position 89 and elsewhere. Other, useful substitutions within SIRPα include one or more of the following: L4V/I, V6I/L, A21V, V27I/L, 131T/S/F, E47V/L, K53R, E54Q, H56P/R, S66T/G, K68R, V92I, F94V/L, V63I, and/or F103V.

In the SIRPαFc fusion protein, the SIRPα component and the Fc component are fused, either directly or indirectly, to provide a single chain polypeptide that may optionally be ultimately produced as a dimer in which the single chain polypeptides are coupled through inter-chain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably have some flexibility to allow the Fc and the SIRP components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G4S sequence (Gly-Gly-Gly-Gly-Ser [SEQ ID NO: 9]) which may repeat as (G4S)n where n is 1, 2, 3 or more, or is based on (Gly)n, (Ser)n, (Ser-Gly)n or (Gly-Ser)n and the like. In another embodiment, the linker is GTELSVRAKPS [SEQ ID NO: 10]. This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The term “CD47⁺” (or CD47+) is used with reference to the phenotype of cells targeted for binding by the present polypeptides. Cells that are CD47⁺ can be identified by flow cytometry using CD47 antibody as the affinity ligand. CD47 antibodies that are labeled appropriately are available commercially for this use (for example, the antibody product of clone B6H112 is available from Santa Cruz Biotechnology). The cells examined for CD47 phenotype can include standard tumour biopsy samples including particularly blood samples taken from the subject suspected of harbouring endogenous CD47⁺ cancer cells. CD47 disease cells of particular interest as targets for therapy with the present fusion proteins are those that “over-express” CD47. These CD47⁺ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

In some dosing regimens and methods provided herein, the SIRPaFc fusion protein is administered as a monotherapy.

In some dosing regimens and methods provided herein, the SIRPaFc fusion protein is administered as part of a combination therapy.

In some embodiments, a combination therapy provided herein includes carfilzomib. Carfilzomib (also known as PR-171) is a structural analogue of the microbial natural product epoxomicin. Carfilzomib selectively inhibits the CTL activity of the 20S proteasome with minimal cross reactivity to the other proteasome classes.

In some embodiments, a combination therapy provided herein includes dexamethasone. Dexamethasone is a synthetic glucocorticoid.

In some embodiments, a combination therapy provided herein includes an anti-CD20 agent. Anti-CD20 agents include, for example, anti-CD20 antibodies. Anti-CD20 antibodies include, for example, rituximab, ocrelizumab, and ofatumumab.

In some embodiments, a combination therapy provided herein includes doxorubicin. Doxorubicin is an anthracycline chemotherapeutic agent. Doxorubicin has the CAS Number 23214-92-8.

In some embodiments, a combination therapy provided herein includes azacitidine. Azacitidine is an analog of cytidine, and is used for the treatment of cancers including myelodysplastic syndrome, myeloid leukemia, and juvenile myelomonocytic leukemia. Azacitidine has the CAS Number 320-67-2.

In some embodiments, a combination therapy provided herein includes venetoclax. Venetoclax is a Bcl-2 inhibitor, and is used for the treatment of cancers including chronic lymphocytic leukemia, small lymphocytic lymphoma, and acute myeloid leukemia. Venetoclax has the CAS Number 1257044-40-8.

A SIRPaFc fusion protein provided herein can be administered in various dosage amounts within the range from about 0.0001 to 100 mg/kg.

In some embodiments, TTI-621 (SEQ ID NO: 7) is administered in the range of 0.01 to 30 mg/kg subject body weight. For example, TTI-621 dosages can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10.0 mg/kg. TTI-621 dosages can also include, for example 0.2-2 mg/kg, 0.7-2 mg/kg, 1-5 mg/kg, 2-5 mg/kg, or 2-10 mg/kg. These dosages of TTI-621 can be administered to a subject, for example, once a week (Q1W), once every two weeks (Q2W), once every three weeks (Q3W), once every four weeks (Q4W), two times a month, once a month, once every two months, or once every three months.

In some embodiments, TTI-622 (SEQ ID NO: 8) is administered in the range of 0.1 to 50 mg/kg subject body weight. For example, TTI-622 dosages can be 0.05 mg/kg, 0.2 mg/kg, 0.4 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. TTI-622 dosages can also include, for example 2-40 mg/kg, 4-40 mg/kg, 5-50 mg/kg, 8-50 mg/kg, 8-40 mg/kg, 8-30 mg/kg, 8-28 mg/kg 10-50 mg/kg, 10-40 mg/kg, 10-30 mg/kg, 10-25 or 10-20 mg/kg. These dosages of TTI-622 can be administered to a subject, for example, once a week (Q1W), once every two weeks (Q2W), once every three weeks (Q3W), once every four weeks (Q4W), two times a month, once a month, once every two months, or once every three months.

In some embodiments, a SIRPaFc fusion protein provided herein [e.g. TTI-622 (SEQ ID NO: 8)] is administered as a “flat” (also referred to as a “fixed”) dose—i.e. the dose is the amount per patient, and the dose does not depend on the mass of the patient. In some embodiments, a SIRPaFc fusion protein such as TTI-622 is administered at a fixed dose of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, or 3600 mg. A fixed dose of SIRPaFc fusion protein may be administered in various regimens. In some embodiments, the dose is administered to a patient weekly (QW), every 2 weeks (Q2W), every 3 weeks (Q3W), or every 4 weeks (Q4W).

In some embodiments, a SIRPaFc fusion protein is administered at a dose between a) a lower level of 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, or 2200 mg and b) an upper level of 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, or 3600 mg, wherein the lower level is a smaller value than the upper level.

SIRPαFc proteins provided herein display negligible binding to red blood cells. There is accordingly no need to account for an RBC “sink” when dosing with SIRPaFc fusion proteins provided herein. Relative to other CD47 blockade drugs that are bound by RBCs, it is estimated that the present SIRPαFc fusions can be effective at doses that are less than half the doses required for drugs that become RBC-bound, such as CD47 antibodies. Moreover, the SIRPαFc fusion proteins provided herein are a dedicated antagonist of the SIRPα-mediated signal, they displays negligible CD47 agonism when binding thereto. There is accordingly no need, when establishing medically useful unit dosing regimens, to account for any stimulation induced by the drug.

Dosing regimens and methods provided herein may be is useful to treat a variety of cancer cells. These include particularly CD47⁺ cancer cells, including liquid (hematological) and solid tumours. Solid tumours can be treated with the dosing regimens and methods provided herein, to reduce the size, number or growth rate thereof and to control growth of cancer stem cells. Such solid tumours include CD47⁺ tumours in bladder, brain, breast, lung, colon, ovary, prostate, liver and other tissues as well. In one embodiment, dosing regimens and methods provided herein can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma. In particular embodiments, dosing regimens and methods provided herein are useful to treat T cell lymphomas that are a very heterogeneous group of lymphoid malignancies divided into cutaneous and peripheral TCL, which themselves are divided into nodal or extranodal types. CTCL derive from skin-homing T cells and consist of mycosis fungoides, Sezary syndrome, primary cutaneous T cell lymphoproliferative disorders, and anaplastic large cell lymphoma. The common features of TCL are aggressive course and poor response to therapy, with the exception of ALK and ALCL.

In some other embodiments, the hematological cancer treated with dosing regimens and methods is a CD47⁺ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with a dosing regimen or method provided herein is a CD47⁺ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

A SIRPαFc fusion protein provided herein can be administered to the subject through any of the routes established for protein delivery, in particular intravenous, intradermal and subcutaneous injection or infusion, or by oral or nasal administration.

In some embodiments, provided herein is a method of treating acute myeloid leukemia (AML) in a patient, comprising administering a SIRPaFc fusion protein comprising the amino acid sequence of SEQ ID NO: 8 to the patient according to a dosing regimen of 8 mg/kg Q1W.

In some embodiments, provided herein is a method of treating acute myeloid leukemia (AML) in a patient, comprising administering a SIRPaFc fusion protein comprising the amino acid sequence of SEQ ID NO: 8 to the patient according to a dosing regimen of 16 mg/kg Q1W.

In some embodiments, provided herein is a method of treating acute myeloid leukemia (AML) in a patient, comprising administering a SIRPaFc fusion protein comprising the amino acid sequence of SEQ ID NO: 8 to the patient according to a dosing regimen of 24 mg/kg Q1W.

In some embodiments, provided herein is a method of treating acute myeloid leukemia (AML) in a patient, comprising administering a SIRPaFc fusion protein comprising the amino acid sequence of SEQ ID NO: 8 to the patient according to a dosing regimen of 32 mg/kg Q1W.

In some embodiments, provided herein is a method of treating multiple myeloma (MM) in a patient, comprising administering a SIRPaFc fusion protein comprising the amino acid sequence of SEQ ID NO: 8 to the patient according to a dosing regimen of 8 mg/kg Q1W.

In some embodiments, provided herein is a method of treating MM in a patient, comprising administering a SIRPaFc fusion protein comprising the amino acid sequence of SEQ ID NO: 8 to the patient according to a dosing regimen of 16 mg/kg Q1W.

In some embodiments, provided herein is a method of treating MM in a patient, comprising administering a SIRPaFc fusion protein comprising the amino acid sequence of SEQ ID NO: 8 to the patient according to a dosing regimen of 24 mg/kg Q1W.

In some embodiments, provided herein is a method of treating MM in a patient, comprising administering a SIRPaFc fusion protein comprising the amino acid sequence of SEQ ID NO: 8 to the patient according to a dosing regimen of 32 mg/kg Q1W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 300 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 600 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 900 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 1200 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 1500 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 1800 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 2100 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 2400 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 2700 mg fixed dose Q1W, Q2W, or Q3W.

In some embodiments, provided herein is a method of treating lymphoma or MM in a patient, comprising administering a SIRPaFc fusion protein comprising the amino acid sequence of SEQ ID NO: 8 to the patient according to a dosing regimen of 3000 mg fixed dose Q1W, Q2W, or Q3W.

In some embodiments, provided herein is a method of treating DLBCL in a patient, comprising administering a SIRPaFc fusion protein comprising the amino acid sequence of SEQ ID NO: 8 to the patient according to a dosing regimen of 8 mg/kg Q1W.

In some embodiments, provided herein is a method of treating DLBCL in a patient, comprising administering a SIRPaFc fusion protein comprising the amino acid sequence of SEQ ID NO: 8 to the patient according to a dosing regimen of 16 mg/kg Q1W.

In some embodiments, provided herein is a method of treating DLBCL in a patient, comprising administering a SIRPaFc fusion protein comprising the amino acid sequence of SEQ ID NO: 8 to the patient according to a dosing regimen of 24 mg/kg Q1W.

In some embodiments, provided herein is a method of treating DLBCL in a patient, comprising administering a SIRPaFc fusion protein comprising the amino acid sequence of SEQ ID NO: 8 to the patient according to a dosing regimen of 32 mg/kg Q1W.

In some embodiments, provided herein is a method of treating DLBCL in a patient, comprising administering a SIRPaFc fusion protein comprising the amino acid sequence of SEQ ID NO: 8 to the patient according to a dosing regimen of 8 mg/kg Q1W.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 8 mg/kg Q2W. Optionally, the solid tumor cancer is ovarian cancer.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 16 mg/kg Q2W. Optionally, the solid tumor cancer is ovarian cancer.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 24 mg/kg Q2W. Optionally, the solid tumor cancer is ovarian cancer.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 32 mg/kg Q2W. Optionally, the solid tumor cancer is ovarian cancer.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 40 mg/kg Q2W. Optionally, the solid tumor cancer is ovarian cancer.

In some embodiments, provided herein is a method of treating a solid tumor cancer in a patient, comprising administering a SIRPaFc fusion protein comprising the amino acid sequence of SEQ ID NO: 8 to the patient according to a dosing regimen of 48 mg/kg Q2W. Optionally, the solid tumor cancer is ovarian cancer.

In some embodiments, provided herein is a method of treating AML in a patient, comprising administering a combination therapy of a SIRPaFc fusion protein and azacitidine to the patient, wherein the SIRPaFc fusion protein comprises the amino acid sequence of SEQ ID NO: 8 and is administered at a dosing regimen of 8 mg/kg Q1W. Optionally, the AML is TP53-mutated AML.

In some embodiments, provided herein is a method of treating AML in a patient, comprising administering a combination therapy of a SIRPaFc fusion protein and azacitidine to the patient, wherein the SIRPaFc fusion protein comprises the amino acid sequence of SEQ ID NO: 8 and is administered at a dosing regimen of 16 mg/kg Q1W. Optionally, the AML is TP53-mutated AML.

In some embodiments, provided herein is a method of treating AML in a patient, comprising administering a combination therapy of a SIRPaFc fusion protein and azacitidine to the patient, wherein the SIRPaFc fusion protein comprises the amino acid sequence of SEQ ID NO: 8 and is administered at a dosing regimen of 24 mg/kg Q1W. Optionally, the AML is TP53-mutated AML.

In some embodiments, provided herein is a method of treating AML in a patient, comprising administering a combination therapy of a SIRPaFc fusion protein and azacitidine to the patient, wherein the SIRPaFc fusion protein comprises the amino acid sequence of SEQ ID NO: 8 and is administered at a dosing regimen of 32 mg/kg Q1W. Optionally, the AML is TP53-mutated AML.

In some embodiments, provided herein is a method of treating AML in a patient, comprising administering a combination therapy of a SIRPaFc fusion protein and azacitidine to the patient, wherein the SIRPaFc fusion protein comprises the amino acid sequence of SEQ ID NO: 8 and is administered at a dosing regimen of 300 mg fixed, 600 mg fixed, 900 mg fixed, 1200 mg fixed, 1500 mg fixed, 1800 mg fixed, 2100 mg fixed, 2400 mg fixed, 2700 mg fixed, or 3000 mg fixed. Optionally, the SIRPaFc fusion protein is administered Q1W, Q2W, Q3W, or Q4W. Optionally, the AML is TP53-wildtype AML.

In some embodiments, provided herein is a method of treating AML in a patient, comprising administering a combination therapy of a SIRPaFc fusion protein, azacytidine, and venetoclax to the patient, wherein the SIRPaFc fusion protein comprises the amino acid sequence of SEQ ID NO: 8 and is administered at a dosing regimen of 8 mg/kg Q1W. Optionally, the AML is TP53-wildtype AML.

In some embodiments, provided herein is a method of treating AML in a patient, comprising administering a combination therapy of a SIRPaFc fusion protein, azacytidine, and venetoclax to the patient, wherein the SIRPaFc fusion protein comprises the amino acid sequence of SEQ ID NO: 8 and is administered at a dosing regimen of 16 mg/kg Q1W. Optionally, the AML is TP53-wildtype AML.

In some embodiments, provided herein is a method of treating AML in a patient, comprising administering a combination therapy of a SIRPaFc fusion protein, azacytidine, and venetoclax to the patient, wherein the SIRPaFc fusion protein comprises the amino acid sequence of SEQ ID NO: 8 and is administered at a dosing regimen of 24 mg/kg Q1W. Optionally, the AML is TP53-wildtype AML.

In some embodiments, provided herein is a method of treating AML in a patient, comprising administering a combination therapy of a SIRPaFc fusion protein, azacytidine, and venetoclax to the patient, wherein the SIRPaFc fusion protein comprises the amino acid sequence of SEQ ID NO: 8 and is administered at a dosing regimen of 32 mg/kg Q1W. Optionally, the AML is TP53-wildtype AML.

In some embodiments, provided herein is a method of treating AML in a patient, comprising administering a combination therapy of a SIRPaFc fusion protein, azacytidine, and venetoclax to the patient, wherein the SIRPaFc fusion protein comprises the amino acid sequence of SEQ ID NO: 8 and is administered at a dosing regimen of 300 mg fixed, 600 mg fixed, 900 mg fixed, 1200 mg fixed, 1500 mg fixed, 1800 mg fixed, 2100 mg fixed, 2400 mg fixed, 2700 mg fixed, or 3000 mg fixed. Optionally, the SIRPaFc fusion protein is administered Q1W, Q2W, Q3W, or Q4W. Optionally, the AML is TP53-wildtype AML.

In some embodiments, provided herein is a method of treating multiple myeloma (MM) in a patient, comprising administering a combination therapy of a SIRPaFc fusion protein, carfilzomib, and dexamethasone to the patient, wherein the SIRPaFc fusion protein comprises the amino acid sequence of SEQ ID NO: 8 and is administered at a dosing regimen of 8 mg/kg Q1W. Optionally, the MM is relapsed and/or refractory (R/R) MM.

In some embodiments, provided herein is a method of treating MM in a patient, comprising administering a combination therapy of a SIRPaFc fusion protein, carfilzomib, and dexamethasone to the patient, wherein the SIRPaFc fusion protein comprises the amino acid sequence of SEQ ID NO: 8 and is administered at a dosing regimen of 16 mg/kg Q1W. Optionally, the MM is R/R MM.

In some embodiments, provided herein is a method of treating multiple myeloma (MM) in a patient, comprising administering a combination therapy of a SIRPaFc fusion protein, carfilzomib, and dexamethasone to the patient, wherein the SIRPaFc fusion protein comprises the amino acid sequence of SEQ ID NO: 8 and is administered at a dosing regimen of 10 mg/kg Q2W. Optionally, the MM is R/R MM.

In some embodiments, provided herein is a method of treating diffuse large B cell lymphoma (DLBCL) in a patient, comprising administering a combination therapy of a SIRPaFc fusion protein and anti-CD20 targeting agent to the patient, wherein the SIRPaFc fusion protein comprises the amino acid sequence of SEQ ID NO: 8 and is administered at a dosing regimen of 8 mg/kg Q1W for 4 weeks, then 18 mg/kg Q3W. Optionally, the DLBCL is CD20+DLBCL.

In some embodiments, provided herein is a method of treating DLBCL in a patient, comprising administering a combination therapy of a SIRPaFc fusion protein and anti-CD20 targeting agent to the patient, wherein the SIRPaFc fusion protein comprises the amino acid sequence of SEQ ID NO: 8 and is administered at a dosing regimen of 16 mg/kg Q1W for 4 weeks, then 28 mg/kg Q3W. Optionally, the DLBCL is CD20+DLBCL.

In some embodiments, provided herein is a method of treating ovarian cancer in a patient, comprising administering a combination therapy of a SIRPaFc fusion protein and pegylated liposomal doxorubicin (PLD) to the patient, wherein the SIRPaFc fusion protein comprises the amino acid sequence of SEQ ID NO: 8 and is administered at a dosing regimen of 12 mg/kg Q1W for 4 weeks, then 12 mg/kg Q2W. Optionally, the ovarian cancer is platinum-resistant ovarian cancer.

In some embodiments, provided herein is a method of treating ovarian cancer in a patient, comprising administering a combination therapy of a SIRPaFc fusion protein and PLD to the patient, wherein the SIRPaFc fusion protein comprises the amino acid sequence of SEQ ID NO: 8 and is administered at a dosing regimen of 24 mg/kg Q1W for 4 weeks, then 24 mg/kg Q2W. Optionally, the ovarian cancer is platinum-resistant ovarian cancer.

In some embodiments, provided herein is a method of treating ovarian cancer in a patient, comprising administering a combination therapy of a SIRPaFc fusion protein and PLD to the patient, wherein the SIRPaFc fusion protein comprises the amino acid sequence of SEQ ID NO: 8 and is administered at a dosing regimen of 48 mg/kg Q1W for 4 weeks, then 48 mg/kg Q2W. Optionally, the ovarian cancer is platinum-resistant ovarian cancer.

In some embodiments, provided herein is a method of treating ovarian cancer in a patient, comprising administering a combination therapy of a SIRPaFc fusion protein and PLD to the patient, wherein the SIRPaFc fusion protein comprises the amino acid sequence of SEQ ID NO: 8 and is administered at a dosing regimen of 48 mg/kg Q2W. Optionally, the ovarian cancer is platinum-resistant ovarian cancer.

Incorporated by reference herein for all purposes is the content of U.S. Provisional Patent Application No. 63/345,693, filed May 15, 2022, and U.S. Provisional Patent Application No. 63/492,121, filed Mar. 24, 2023.

The following examples of specific aspects for carrying out the present invention are offered for illustrative purposes only, and are not intended to limit the scope of the present invention in any way

### EXAMPLES

In order that this invention may be better understood, the following examples are set forth. These examples are for purposes of illustration only and are not to be construed as limiting the scope of the invention in any manner.

### Example 1: Clinical Study of TTI-622 in Patients with Advanced Hematologic Malignancies, Including Multiple Myeloma

The objective of this study is to evaluate the safety and preliminary efficacy of TTI-622 monotherapy and in combination with carfilzomib and dexamethasone in patients with relapsed and/or refractory (R/R) multiple myeloma (MM).

This is a multi-center Phase 1a/1b study. Phase 1a was designed to determine the MTD, pharmacokinetics (PK), pharmacodynamics, and preliminary antitumor activity of QW, Q2W, and Q3W single-agent TTI-622 in R/R lymphoma using a 3+3 dose escalation schema Phase 1b, will determine the safety and recommended dose of TTI-622 to be given as single agent and in combination with carfilzomib+dexamethasone in R/R MM and will evaluate the preliminary efficacy. Secondary objectives are to further characterize the safety, PK and immunogenicity of TTI-622 when combined with carfilzomib+dexamethasone. Patients will be enrolled in 5 separate cohorts: 3 cohorts will explore different doses and administration schedules of TTI-622 (8 mg/kg QW, 16 mg/kg QW and 10 mg/kg Q2W) combined with the approved dose of carfilzomib+dexamethasone. (Carfilzomib and dexamethasone are administered in 28-day cycles. Carfilzomib is administered on days 1, 8, and 15 of the 28-day cycle; it is administered at 20 mg/m2 IV on Cycle 1 Day 1, and if tolerated, then at 70 mg/m2 IV starting on Cycle 1 Day 8, and subsequent doses thereafter. Dexamethasone is administered on days 1, 8, 15, and 22 of the 28-day cycle; it is administered at 40 mg orally or by IV.) 2 cohorts will explore different doses of TTI-622 monotherapy (8 mg/kg and 16 mg/kg QW). Cohorts will open in a staggered manner. In each cohort 3 patients will be dosed and followed for 28 days (21 days in the monotherapy) before expanding enrollment to additional 27 patients to explore efficacy. Eligibility criteria include: relapse or progression following ≥3 prior lines of therapy (including a proteasome inhibitor, an immunomodulatory drug, and an anti-CD38 antibody), carfilzomib-refractory progressive and measurable disease per IMWG at study entry; age ≥18 years; ECOG performance status ≤2; adequate organ functions; no known CNS involvement; no prior anti-CD47 or anti-SIRPα therapy.

Endpoints of the study include, for example, dose limiting toxicities (DLTs), frequency and severity of adverse events, overall response rate, disease control rate, time to response, duration of response, progression-free survival, minimal residual disease status, physical examination results, vital sign measurements, electrocardiogram results, ECOG performance status, laboratory evaluations, anti-drug antibodies against TTI-622, assessment of single-dose PK of TTI-622, PK after repeated TTI-622 administration, alone or in combination with carfilzomib and dexamethasone.

### Example 2: Clinical Study of TTI-622 in Patients with Advanced Hematologic Malignancies, Including Diffuse Large B Cell Lymphoma

The objective of this study is to evaluate the safety and preliminary efficacy of TTI-622 in combination with an anti-CD20 targeting agents in patients with CD20+ relapsed and/or refractory (R/R) diffuse large B cell lymphoma (DLBCL).

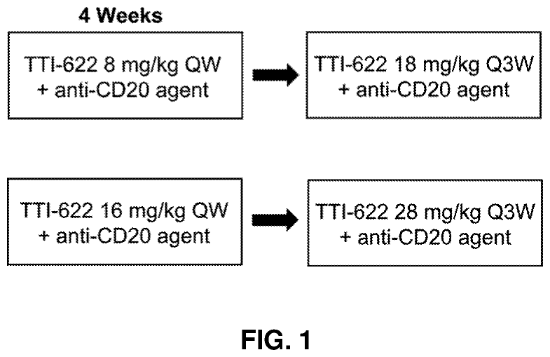

This is a multi-center Phase 1a/1b study. Phase 1a was designed to determine the MTD, pharmacokinetics (PK), pharmacodynamics, and preliminary antitumor activity of QW, Q2W, and Q3W single-agent TTI-622 in R/R lymphoma using a 3+3 dose escalation schema Phase 1b, ongoing, will determine the safety, recommended dose and preliminary efficacy of TTI-622 in combination with select approved anticancer treatments for patients with hematological malignancies including, but not limited to anti-CD20 therapy in patients with CD20+ R/R DLBCL. Secondary objectives are to further characterize safety, PK and immunogenicity of TTI-622 when combined with approved therapies. Patients will be enrolled in 2 cohorts exploring different doses of TTI-622 (8 mg/kg QW for 4 weeks, then 18 mg/kg Q3W and 16 mg/kg QW for 4 weeks, then 28 mg/kg Q3W) in combination with anti-CD20 therapy (The anti-CD20 agent Rituxan is administered weekly at 375 mg/m2 for up to 8 doses). A schematic of these dosing regimens is shown in FIG. 1. Cohorts will open in a staggered manner. In each cohort 3 patients will be dosed and followed for 28 days before expanding enrolment to additional 27 patients per cohort to explore efficacy. Key eligibility criteria include: age ≥18 years; relapsed and/or refractory disease after ≥1 prior line of therapy; not eligible for or have progressed after high dose chemotherapy (HDT)/auto-SCT; ≥1 site of measurable disease per the Lugano 2014 classification; ECOG PS ≤2; adequate organ functions, no known CNS involvement; no prior anti-CD47 or anti-SIRPα therapy.

Endpoints of the study include, for example, dose limiting toxicities (DLTs), frequency and severity of adverse events, overall response rate, disease control rate, time to response, duration of response, progression-free survival, physical examination results, vital sign measurements, electrocardiogram results, ECOG performance status, laboratory evaluations, anti-drug antibodies against TTI-622, assessment of single-dose PK of TTI-622, PK after repeated TTI-622 administration, alone or in combination with anti-CD20 targeting agents.

### Example 3: Clinical Study of TTI-621 in Combination with Doxorubicin in Patients with Unresectable or Metastatic High-Grade Leiomyosarcoma

The objective of this study is to evaluate the safety and clinical activity of TTI-621 in combination with doxorubicin in patients with unresectable or metastatic high-grade leiomyosarcoma (LMS).

This is a Phase 1/2, open-label study of TTI-621 in combination with doxorubicin in patients with anthracycline-naïve disease. The Phase 1 dose escalation evaluates doses of TTI-621 (0.2 to 2.0 mg/kg) in combination with doxorubicin at 75 mg/m² in patients with high-grade soft tissue sarcomas. Expansion cohorts will evaluate TTI-621 (0.2 and 2.0 mg/kg) with doxorubicin in patients with LMS, with pathology confirmed at a central laboratory. Doxorubicin is administered on Day 1 and TTI-621 is administered on Days 1 and 8 of 21-day cycles for to up six cycles; patients continue on TTI-621 monotherapy (Days 1 and 15 of 28-day cycles) until disease progression. The primary goals of this study are evaluation of safety of TTI-621 administered in combination with standard-of-care doxorubicin and to further evaluate clinical activity (ORR, PFS, OS), safety, PK and patient-reported quality of life in the LMS subpopulation. The dose escalation portion of the study has been completed without DLT.

Endpoints of the study include, for example, overall safety profile of TTI-621 in combination with doxorubicin and as a monotherapy, objective response rate, progression-free survival, overall survival, disease control rate, duration of response, duration of disease control, time to radiologic progression, time to new metastases, time to worsening of ECOG performance status, time to worsening of patient-reported quality of life assessments.

It is claimed:

1. A method of treating a cancer in a patient, comprising administering a SIRPaFc fusion protein to the patient according to a dosing regimen of 8 mg/kg, 10 mg/kg, 16 mg/kg, 18 mg/kg, 24 mg/kg or 28 mg/kg Q1W, Q2W, or Q3W.

2. A method of treating a cancer in a patient, comprising administering a SIRPaFc fusion protein to the patient according to a dosing regimen of 8 mg/kg Q1W for 4 weeks followed by 18 mg/kg Q3W.

3. A method of treating a cancer in a patient, comprising administering a SIRPaFc fusion protein to the patient according to a dosing regimen of 16 mg/kg Q1W for 4 weeks followed by 28 mg/kg Q3W.

4. A method of treating a cancer in a patient, comprising administering a SIRPaFc fusion protein to the patient according to a dosing regimen of 8 mg/kg Q1W, 18 mg/kg Q3W, 16 mg/kg QW, or 28 mg/kg Q3W.

5. The method of any one of claims 1-4 further comprising administering an anti-CD20 agent to the patient.

6. A method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 8 mg/kg Q1W for 4 weeks followed by 18 mg/kg Q3W.

7. A method of treating a cancer in a patient, the method comprising administering a combination therapy of a SIRPaFc fusion protein and anti-CD20 agent to the patient, the method comprising administering the anti-CD20 agent at 375 mg/m2 Q1W to the patient for up to eight doses, and administering the SIRPaFc fusion protein to the patient according to a dosing regimen of 16 mg/kg Q1W for 4 weeks followed by 28 mg/kg Q3W.

8. The method of any one of claims 5-7 wherein the anti-CD20 agent is rituximab.

9. A method of treating a cancer in a patient, the method comprising administering a combination therapy of a SIRPaFc fusion protein, carfilzomib, and dexamethasone to the patient for N cycles, wherein each cycle is 28 days and the SIRPaFc fusion protein is administered at 8 mg/kg or 16 mg/kg on days 1, 8, 15, and 22 of the 28 day cycle, carfilzomib is administered at 20 mg/m2 or 70 mg/mg2 on days 1, 8, and 15 of the 28 day cycle, dexamethasone is administered at 40 mg on days 1, 8, 15, and 22 of the 28 day cycle.

10. The method of claim 9, wherein N is 1, 2, 3, 4, 5, 6, 7, or 8 cycles.

11. A method of treating a cancer in a patient, comprising administering a SIRPaFc fusion protein to the patient according to a dosing regimen of 0.2 mg/kg, 0.7 mg/kg, or 2.0 mg/kg Q2W.

12. A method of treating a cancer in a patient, the method comprising a first regimen and a second regimen, wherein the first regimen comprises administering a combination therapy of a SIRPaFc fusion protein and doxorubicin for to the patient for N cycles, wherein each cycle is 21 days and the SIRPaFc fusion protein is administered on day 1 and 8 of the 21 day cycle and doxorubicin is administered on day 1 of the 21 day cycle, and wherein N is 2, 3, 4, 5, 6, 7, or 8 cycles, and wherein the second regimen follows the first regimen and comprises administering a SIRPaFc fusion protein to the patient according to a dosing regimen of Q2W.

13. The method of any one of claims 1-12, wherein the SIRPaFc fusion protein comprises a SIRPa polypeptide comprising the amino acid sequence of SEQ ID NO: 1.

14. The method of any one of claims 1-13, wherein the SIRPaFc fusion protein comprises a SIRPa polypeptide comprising the amino acid sequence of SEQ ID NO: 2.

15. The method of any one of claims 1-14, wherein the SIRPaFc fusion protein comprises the amino acid sequence of SEQ ID NO: 7 or SEQ ID NO: 8.

16. The method of any one of claims 1-13, wherein the SIRPaFc fusion protein comprises a SIRPa polypeptide comprising the amino acid sequence of SEQ ID NO: 1 or a variant thereof having one, two, three, four, or five amino acid substitutions as compared the sequence of SEQ ID NO: 1.

17. The method of any one of claims 1-16, wherein the cancer is a blood cancer or a solid tumor cancer.

18. The method of any one of claims 1-17, wherein the cancer is selected from the group consisting of acute lymphocytic leukemia (ALL); acute myeloid leukemia (AML) and p53 mutated AML; chronic lymphocytic leukemia (CLL); chronic myelogenous leukemia (CML); myeloproliferative disorder/neoplasm (MPDS); myelodysplastic syndrome, lymphoma, T cell lymphoma, Hodgkin's lymphoma, indolent non-Hodgkin's lymphoma, aggressive non-Hodgkin's lymphoma, Burkitt's lymphoma, small cell follicular lymphoma, large cell follicular lymphoma. myeloma, multiple myeloma (MM), giant cell myeloma, heavy-chain myeloma, light chain or Bence-Jones myeloma, sarcoma, soft tissue sarcoma, leiomyosarcoma (LMS), undifferentiated pleomorphic sarcoma, myxofibrosarcoma, dedifferentiated liposarcoma, angiosarcoma, or epithelioid sarcoma.

19. The method of any one of claims 1-18, wherein the SIRPaFc fusion protein is administered for 12 doses or fewer.

20. The method of any one of claims 1-18, wherein the SIRPaFc fusion protein is administered until disease progression.

21. The method of any one of claims 1-20, wherein the patient has CD47-positive cancer cells.

22. A SIRPaFc fusion protein for use to treat a patient according to the method of any one of claims 1-21.

23. Use of a SIRPaFc fusion protein in the manufacture of a medicament for use to treat a patient according to the method of any one of claims 1-21.

24. A kit comprising a SIRPaFc fusion protein and instructions for use according to the method of any one of claims 1-21, and optionally further comprising one or more additional therapeutic agents according the method of any one of claims 1-21.
